(19) United States
(12) Patent Application Publication
(10) Pub. No.: US 2004/0176827 A1
(43) Pub. Date: Sep. 9, 2004

(54) Frozen saline nasal applicator

(76) Inventors: Abby Noma Jacobson, Vero Beach, FL (US); Peter Marion Crane JR., Vero Beach, FL (US)

Correspondence Address: BEUSSE BROWNLEE WOLTER MORA & MAIRE, P. A., 390 NORTH ORANGE AVENUE, SUITE 2500, ORLANDO, FL, 32801, US

(21) Appl. No.: 10/382,407

(22) Filed: Mar. 6, 2003

(51) Int. Cl. A61F 7/12

(52) U.S. Cl. 607/113

(57) ABSTRACT

A method and device for treating nasal cavity irritation, such as symptoms of allergic rhinitis. The device includes a nasal suppository having a plug shape for placement within a nasal cavity. In an embodiment, the suppository is frozen saline solution in a therapeutic concentration. In another embodiment, the device includes a handle, frozen within a portion of the suppository and extending therefrom, to allow grasping and insertion of the suppository in an affected nasal cavity. In yet another embodiment, a leak-resistant molding package having a chamber for accepting a liquid and shaped to conform to the interior of a nasal passage, is provided. The method includes inserting an elongated, conformally shaped plug of a frozen liquid into a nasal cavity; removably lodging the frozen liquid in the nasal cavity, and allowing the frozen liquid to gradually melt within the nasal cavity.

### FIELD OF THE INVENTION

The present invention relates generally to treatment of nasal irritation, and more particularly, to a frozen saline applicator for insertion into a nasal cavity for relieving allergic rhinitis symptoms.

### BACKGROUND OF THE INVENTION

Approximately 40 million people in the United States suffer from a condition known as “allergic rhinitis.” According to the Center for Disease Control, 5 million new cases are diagnosed each year. Allergic rhinitis is the most common chronic disease in humans and generally manifests between ages five and thirty years. Furthermore, according to the Center for Disease Control, in the year 2000, 9 million medical doctor visits were attributed to allergic rhinitis at an approximate cost of 500 million dollars for office visits alone.

It has been reported that the occurrence of allergic rhinitis in Great Britain is 23% of the population, and 36% of the population in Japan suffers from this condition. Similar conditions such as hay fever, allergies, nasal and sinus conditions, and colds can all affect the nasal passages.

Anatomically, allergic rhinitis is defined as an inflammation of the mucous membranes that line the nose and nasal passages. It is magnified by a combination of symptoms which include nasal congestion, nasal obstruction, discharge, sneezing, and facial pain and swelling, membrane dryness, and inability to breathe. It can be “seasonal” such as in hay fever or perinnieal such as in allergic reaction to dust mite feces. It also appears in “episodic” reactions such as in allergies to animal dander.

Current products and treatments that attempt to address these nasal symptoms include allergy shots, injected medications, oral steroids, oral antihistamines, intra-nasal antihistamines, oral decongestants, a wide variety of nasal sprays, nasal strips, and dilating devices. All of these products have drawbacks and shortcomings and are unable to solve the condition for all sufferers. Furthermore, the medications all have published side effects. Allergy shots and injected medications cannot be self-administered and are therefore inconvenient and time consuming, and are not entirely successful in opening swollen nasal cavities.

In an attempt to deal with these shortcomings, various devices and methods have been devised, including those described in the following patents: U.S. Pat. No. 4,749,700 to Weenie, issued Jun. 7, 1988, U.S. Pat. No. 4,778,810 to Wenig, et al., issued Oct. 18, 1988, and U.S. Pat. No. 4,729,997 to Wenig, issued Mar. 8, 1988. In addition, nasal saline sprays have been used to moisturize nasal passages and to dissolve build-up of the nasal mucosa. However, saline solutions alone have not proved satisfactory for the relief of nasal congestion.

Nasal dilators for aiding breathing through the nose are known. However, these devices are not generally effective in relieving nasal congestion and blockage, sinus discomfort and pain, and other cold/allergy symptoms. U.S. Pat. No. 4,414,977 issued to Rezakhany discloses one such nasal dilator. This dilator includes top and bottom rings connected by a rear strut and a front strut, and is placed in the nasal passage. Such a nasal dilator suffers from the drawbacks of being uncomfortable to wear, causing irritation and itching of the nostril, being unsafe to use at night during sleep, and being inconvenient to use when the wearer has nasal drainage. Other nasal dilators are disclosed in U.S. Pat. No. 5,533,499, issued to Johnson, U.S. Pat. No. 5,533,503, issued to Doubek, et al., and U.S. Pat. No. 5,546,929, issued to Muchin. These nasal dilators are flexible strips with spring members that adhere to the bridge of the nose and adhere to the exterior surface of the nose. They can be unsightly, do nothing to eliminate swelling,: and have no moisturizing features. Furthermore, U.S. Pat. No. 5,890,486, issued to Mitra, et al., April 1999, is another truss style nasal dilator held in place by an adhesive substance, and incorporates a thermal element. This product also fails to address swollen membranes dry mucosa or in the nasal passage.

U.S. Pat. No. 4,369,777, issued to Lwoff, et al., Jan. 25, 1983, describes an invention to deliver a stream of heated, humidified air to the nasal mucosa. This product does address the dry mucous membrane but will intensify the swollen tissues through the application of heat, thereby making breathing more difficult. U.S. Pat. No. 5,693,077, issued to Friedman, Dec. 2, 1997, calls for the application of cold or frozen water or saline in small bags, plastic tubes or metal tubes, applied intra-orally. This invention fails to apply the needed relief to the nasal passages directly and can be uncomfortable when used in metal tubes. U.S. Pat. No. 5,527,351, issued to Friedman, Jun. 18, 1996 describes a similar invention having similar drawbacks as the '077 patent.

Thus, there is a need in the art to provide a device that reduces nasal swelling and provides moisture in the nasal cavity. In particular, there is a need to provide a device that reduces nasal swelling through the direct application of cold to inflamed nasal passages, and provides a safe and inexpensive alternative to medications and inhalants. In addition, there is a need to provide a device that offers a more comfortable method of opening the nasal passages than is currently available in conventional nasal dilators, and less unsightly than external nasal strips. Further, there is a need in the art to provide a device that can be used, in a non-interfering manner, in conjunction with other currently accepted forms of medication and treatment, and a device which provides a safe and effective alternative to corrective surgery. Additionally, there is a need in the art to provide a device which can be produced in various sizes to meet the needs of all potential users, both adults and children, can be used on an individual dosage basis, and is disposable to prevent the spread of infection and cross contamination.

### SUMMARY OF THE INVENTION

A nasal suppository for treatment of nasal irritation, described herein as including a suppository having a plug of a frozen liquid for placement within a nasal cavity. The nasal suppository may further include a frozen liquid made from a saline solution having a therapeutic concentration. The nasal suppository may also include an elongated handle, positioned within a portion of the suppository and extending from one end of the suppository.

A method for distributing nasal suppositories is described herein as including providing a molding package sealable for forming a leak resistant chamber having a shape conforming to the interior of a nasal passage. The method described herein also includes injecting a liquid into the chamber at an injection site for forming a plug corresponding to the shape of the chamber when the liquid is frozen and sealing the chamber at the injection site to prevent leakage from the chamber. The method may also include positioning an end of an elongated handle axially into a portion of the chamber so that the other end of the elongated handle protrudes from the molding package and then sealing the molding package around the elongated handle.

A method of treatment for nasal cavity irritation is described herein as including inserting an elongated, conformally shaped plug of a frozen liquid into a nasal cavity; removably lodging the frozen liquid in the nasal cavity; and allowing the frozen liquid to gradually melt.

### BRIEF DESCRIPTION OF THE DRAWINGS

The features of the invention believed to be novel are specifically set forth in the appended claims. The features and advantages of the present invention will become apparent from the following detailed description of the invention when read with the accompanying drawings in which:

FIG. 1 shows an embodiment of the frozen saline applicator.

FIG. 2 shows an embodiment of the handle of the frozen saline applicator.

FIG. 3 shows the frozen saline applicator in an individual dosage package.

FIG. 4 shows a number of frozen saline applicators, each contained respective individual dosage packages in a dispensing arrangement.

FIG. 5 shows an embodiment of the frozen saline applicator positioned in a human nasal cavity.

### DETAILED DESCRIPTION OF THE INVENTION

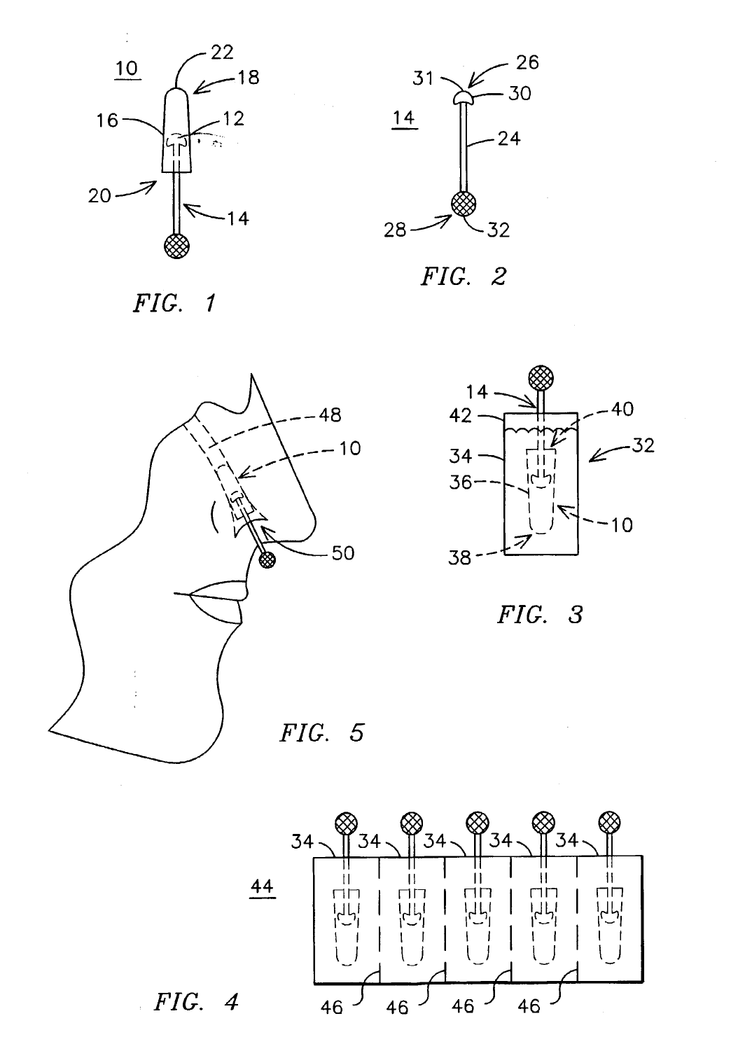

FIG. 1 shows an embodiment of the frozen saline applicator 10 of the invention. Generally, the applicator 10 comprises nasal suppository 12 and may include a handle 14. In the context of the invention, the term suppository is intended to mean a dissolvable plug, which may or may not include a medication, for placement in a nasal cavity for a therapeutic purpose. The nasal suppository 12 can be generally shaped as an elongated plug to allow insertion into a nasal cavity for treatment of nasal cavity irritation, such as allergic rhinitus. In particular, the suppository 12 can include a generally elongate, tapering shape 16, having a relatively smaller cross section at a proximal end 18, and a relatively larger cross section at a distal end 20. In an aspect of the invention, the proximal end 18 is hemispherical 22 to ease insertion into the nasal cavity and to minimize irritation of sensitive tissues, such as nasal mucosa. In another aspect, the suppository 12 is conformally shaped to match the interior of a human nasal cavity. For example, the suppository 12 may be adapted for different sizes and shapes to accommodate variability in the size and shape of nasal cavities in humans, such as nasal cavity size variability between infants, children, and adults.

In a further aspect of the invention, the suppository 12 is formed from a frozen liquid, such as water or a saline solution in a therapeutic concentration. The suppository 12 may or may not include a medication, such as menthol, a decongestant, an antihistamine, a steroid or other known medications to treat nasal irritations or symptoms of rhinitis or sinusitis. Advantageously, the frozen suppository 12 provides relief from inflamed tissues by reducing nasal swelling through the direct application of a cold element to inflamed nasal passages. In addition, the frozen suppository 12 melts gradually after being inserted into an affected nasal cavity, thereby providing moisture to dry tissues. Accordingly, the frozen suppository 12, helps relieve the swelling and nasal passage dryness associated with rhinitis, such as allergic rhinitus, without requiring the use of orally or nasally administered medications as in conventional treatments.

FIG. 2 shows an embodiment of the handle 14 of the frozen saline applicator 10. The suppository 12 is mounted on the handle 14 for facilitating grasping of the applicator 10 and positioning of the suppository 12 in the nasal cavity. In an aspect of the invention, the handle may be removable. In another aspect of the invention, the handle 14 generally comprises an elongated shaft 24, having a proximal end 26 axially embedded, along an elongate direction of the suppository 12, in a portion of the suppository 12 near the distal end 22. The distal end 28 of the shaft 24 extends from the distal end 22 of the suppository 12 to allow grasping of the handle 10. For example, the distal end 28 of the shaft 24 may protrude from the distal end 22 of the suppository 12 by approximately 2 inches (5.08 centimeters). In an embodiment, the handle 14 may include a head 30 at a proximal end 26 of the shaft 24 for anchoring the handle 14 within the suppository 12. In a further aspect, the head 30 can be is mushroom-shaped, with the convex face 31 of the mushroom-shaped head 30 oriented towards the proximal end 18 of the suppository 12 to provide anchoring of suppository 12. In another embodiment, the handle 14 includes a grip 32 at a distal end 28 to provide a graspable surface for holding and positioning the applicator 10. For example, the grip 32 may include a disk mounted, or formed, on the shaft 24, which can be grasped between a thumb and forefinger, for example, for handling the applicator. In another aspect, the disk may include a gripping surface, such as checkering, on one or both sides of the disk.

Turning now to the dispensing aspects of the invention, FIG. 3 shows the frozen. saline applicator 10 in an individual dosage package 32. Generally, the individual dosage package 32 includes a leak resistant molding package 34 comprising an elongate chamber 36 for accepting a nasal passage therapeutic liquid, such as a saline solution. In an aspect of the invention, the chamber 36 has a shape to conform to the interior of a nasal passage, so that when a liquid is injected into the chamber 36 and subsequently frozen, the frozen liquid assumes the shape of the chamber 36 and, correspondingly conforms to the interior of a nasal passage. For example, the chamber 36 may have a generally tapering shape, as described previously regarding the suppository 12. In other aspects, the proximal end 36 of the chamber is hemispherical, and the chamber 36 may be conformally shaped to match the interior of a human nasal cavity. In addition, the chamber 36 may be adapted to different sizes and shapes to accommodate variability in the size and shape of nasal cavities in humans, such as nasal cavity size variability between infants, children, and adults.

The handle 14, for a suppository 12 to be formed in the chamber 36, extends axially along an elongate direction of the chamber 36, into a portion of the chamber 36 and protrudes from an end of the molding package 34. A liquid, such as a therapeutic saline solution, is introduced into the chamber 36 so that the chamber 36 is substantially filled. Accordingly, when frozen, the fluid forms a suppository 12 around the handle 14 corresponding to the shape of the chamber 36. Therefore, the shape of the chamber 36 determines the shape of the frozen suppository 12. In aspect of the invention, the package 34 is separable so that a frozen suppository 12 may be extracted from the package 34 for application in an affected nasal cavity. For example, a pull away tab 42 may be provided so that the package may be peeled into two halves to release the suppository 12.

FIG. 4 shows a number of frozen saline applicators 10, each contained in respective individual dosage packages 34 in a dispensing arrangement 44. In an aspect of the invention, the individual packages 34 may be removably attached to one another for ease in dispensing. For example, the individual packages 34 may by attached along elongate sides, and perforations 46 may be provided to allow individual packages 34 to be easily removed from an attachment to another package 34. Accordingly, a convenient means for dispensing the saline applicators 10 is provided. In an aspect of the invention, the dispensing arrangement 44 can be purchased and stored at room temperature if desired, so that the saline solution in the chamber 36 remains in a liquid form. When use of the saline applicator 10 is indicated, one or more of the packages 34 can be placed in a freezer, for example, to freeze the liquid saline solution in the chamber 36. Once the saline solution is frozen, an individual package 34 can be separated from the dispensing arrangement 44 and the frozen saline applicator 10 can be removed from the package 34. The released frozen saline applicator 10 can then be inserted into an affected nasal cavity for relief from nasal cavity irritation, such as inflammation and dryness associated from allergic sinusitis.

FIG. 5 shows the frozen saline applicator 10 positioned in a human nasal cavity 48. After removal from the package, the handle 14 is grasped and used to gently insert the suppository 12, proximal end 18 first, through the nostril 50 and into the affected nasal cavity 48, until the suppository 12 is comfortably lodged in the nasal cavity 48. Advantageously, the frozen suppository 12 helps to shrink swollen nasal cavity membranes and, as the suppository 12 melts, provides moisture to ease dryness of the nasal cavity. Accordingly, as the suppository 12 melts, it can be repositioned in the nasal cavity as required. When the suppository 12 melts sufficiently so that it can no longer remain lodged in the nasal cavity 48, the saline applicator 10 is removed and discarded. A new frozen applicator 10 may then be inserted if needed.

In an aspect of the invention, the handle 14 may be removable from the suppository 12 after insertion in the nasal cavity 48. For example, the handle 14 may be formed without an anchoring head 30, and may be coated with a release agent to facilitate removal from the suppository 12. In another aspect of the invention, the saline applicator does not include a handle 14, but may be positioned in the nasal cavity using a finger or other suitable applicator. If there is a concern that the frozen suppository 12 may be drawn into a sinus cavity, such as if a sharp inhalation is drawn through the nose, a piece of string, for example, frozen into a portion of the of the suppository 12 and extending therefrom, may be used, instead of the handle 14, to allow removal of the suppository 12 from the nasal cavity 48.

While only certain preferred features of the invention have been shown by way of illustration, many modifications and changes will occur to those skilled in the art. It is, therefore, to be understood that the present claims are intended to cover all such modifications and changes, which fall within the true spirit of the invention.

What is claimed is:

1. A nasal suppository for treatment of nasal irritation, the suppository comprising a plug of a frozen liquid for placement within a nasal cavity.

2. The nasal suppository of claim 1, wherein the frozen liquid comprises a saline solution having a therapeutic concentration.

3. The nasal suppository of claim 1, wherein the frozen liquid comprises a medication for the treatment of symptoms associated with rhinitis.

4. The nasal suppository of claim 1, wherein the plug assumes an elongate tapering shape having a generally hemispherical proximal end.

5. The nasal suppository of claim 1, wherein the suppository is conformally shaped to match the interior of a human nasal cavity.

6. The nasal suppository of claim 1, further comprising an elongated handle, positioned axially within a portion of the plug and extending from a distal end thereof.

7. The nasal suppository of claim 6, wherein the handle further comprises a head at a proximal end of the applicator for anchoring the handle within the portion of the plug.

8. The nasal suppository of claim 7, wherein the head is generally mushroom-shaped.

9. The nasal suppository of claim 6, wherein the handle further comprises a grip at a distal end.

10. The nasal suppository of claim 9, wherein the grip is generally disk shaped.

11. The nasal suppository of claim 1, further comprising a string, positioned axially within a portion of the plug, and extending from a distal end thereof.

12. A method for distributing nasal suppositories comprising:
providing a molding package sealable for forming a leak resistant chamber having a shape conforming to the interior of a nasal passage;
injecting a liquid into the chamber at an injection site for forming a plug corresponding to the shape of the chamber when the liquid is frozen; and
sealing the chamber at the injection site to prevent leakage from the chamber.

13. The method of claim 12, further comprising:
positioning a first end of an elongated handle axially into a portion of the chamber so that a second end of the elongated handle protrudes from the molding package; and
sealing the molding package around the elongated handle.

14. The method of claim 12, further comprising:
positioning a first end of a string into a portion of the chamber so that a second end of the string protrudes from the molding package; and
sealing the molding package around the string.

15. The method of claim 12, further comprising removably connecting a plurality of molding packages.

16. The method of claim 12, wherein the liquid is a saline solution comprising a therapeutic concentration.

17. The method of claim 12, wherein the frozen liquid comprises a medication for the treatment of symptoms associated with rhinitis.

18. A method of treatment for nasal cavity irritation comprising:
inserting a conformally shaped plug of a frozen liquid into a nasal cavity;
removably lodging the frozen liquid in the nasal cavity; and
allowing the frozen liquid to gradually melt.

19. The method of claim 18, wherein the frozen liquid is a saline solution comprising a therapeutic concentration.

20. The method of claim 18, wherein the frozen liquid comprises a medication for the treatment of symptoms associated with rhinitis.
